(19) United States
(12) Patent Application Publication
(10) Pub. No.: US 2023/0234032 A1
(43) Pub. Date: Jul. 27, 2023

(54) FILTER MEDIUM FOR AIR AND WATER PURIFICATION AND DISINFECTION

(71) Applicant: Helvyatech GmbH, Zug (CH)

(72) Inventor: Filippo BARBARO, Venezia (IT)

(21) Appl. No.: 18/011,204

(22) PCT Filed: Jun. 4, 2021
(86) PCT No.: PCT/EP2021/065002

§ 371 Date: Dec. 19, 2022

(30) Foreign Application Priority Data
Jun. 19, 2020 (CH) 00736/20

(51) Int. Cl. B01J 21/06; B01D 39/20; B01D 53/88; C02F 1/32; C02F 1/72; A61L 9/20; B01J 23/50; B01J 23/30; B01J 31/06; B01J 31/38; B01J 35/06; B01J 35/02; B01J 35/00

(52) U.S. Cl. CPC B01J 21/063; B01D 39/2041; B01D 53/885; C02F 1/325; C02F 1/725; A61L 9/205; B01J 23/50; B01J 23/30; B01J 31/06; B01J 31/38; B01J 35/06; B01J 35/023; B01J 35/004; B01D 2239/0492; B01D 2239/0618; B01D 2239/0258; B01D 2239/025; B01D 2239/0442; B01D 2239/1233; B01D 2259/804; B01D 2259/808; B01D 2255/802; B01D 2255/104; B01D 2255/20776; B01D 2255/20707; B01D 2257/91; B01D 2259/4541; B01D 2255/9202; C02F 2305/10; C02F 2305/08; C02F 2201/3226; C02F 2201/3222; C02F 2303/04; A61L 2209/12; A61L 2209/14; A62B 23/02

(57) ABSTRACT

The present invention relates to a filter medium (10) for air and/or water cleaning, comprising a semiconductor photocatalytic material (14) and a light energy source (15) for radiating light provided to activate photocatalytic reactions of the semiconductor photocatalytic material (14). The light energy source (15) is configured as a support (16) for the semiconductor photocatalytic material (14). The filter medium (10) can be incorporated into a filter unit (100).

### TECHNICAL DOMAIN OF THE INVENTION

The present invention relates to a filter medium for air and water purification and disinfection, in particular to a photocatalytic filter medium for removal of organic and inorganic pollutants. In particular, the present invention relates to a photocatalytic filter medium comprising nanofibers showing photocatalytic activity and with an incorporated light energy source used for cleaning air and/or water from contaminants such as pollutant chemicals, microorganism or pharmaceutical residual. In particular, the filter medium can remove organic airborne pathogen of many types, including viruses, bacteria, mycotoxin, fungi, spores and allergen particles. Such a filter medium can be accommodated as a filter unit into an air and/or water cleaner provided with a lightproof casing. Such a filter unit can be provided in an air circulation duct, a personal breathing device such as an anti-smog mask or as a stand-alone filtering device.

### BACKGROUND ART

Filters applying photocatalysis as the basic principle are well known. A filter medium comprising semiconductor photocatalytic material such as nanofibers loaded or doped with photocatalysts in form of nanoparticles uses incident irradiation in the UV or visible region of the spectrum for a charge transfer mechanism. On irradiation of light, an absorption of photon takes place in the semiconductor material and excites an electron from the valence band to the conduction band if the photon energy equals or is greater than the band gap of the semiconductor photocatalytic material. In particular, electrons are excited from the valence band of the light-sensitive semiconductor, leaving a hole behind, to the conduction band. These photo-induced charge carriers proceed forming reactive intermediates, in particular oxidizing and reducing species as well as hydroxyl radicals and super oxide radicals, which attack chemicals absorbed or diffused on the surface of the photocatalysts during the lifetime of the reactive intermediate. Another reaction taking place in the absence of suitable scavenger exists in recombination of electron and hole pair producing thermal energy. However, only molecules in direct contact with the photocatalytic surface will participate in the photocatalytic reactions.

Nanoparticles used as photocatalysts such as titanium dioxide (TiO₂) and/or tungsten trioxide (WO₃) in filter media show defined characteristic properties such as a large specific surface area, high stability, low toxicity, simple synthetic routes, good absorptivity, electronic mobility and cost-efficiency.

Titanium dioxide is a most widely explored light-sensitive semiconductor due to its superior photocatalytic activity by absorbing electromagnetic radiation near UV region, its optical and electrical properties, nontoxicity, excellent physical and chemical stability, low cost and high availability. TiO₂ nanoparticles can be used with different phase compositions, crystallinities such as anatase and/or rutile and surface areas showing variation in properties and photocatalytic performance. One example of a photocatalytic nanoparticle is P25 from Degussa (Evonik) used as a light-sensitive semiconductor in photocatalysis in environmental pollutants treatment. P25 is a biphasic TiO₂ with high photocatalytic activity containing more than 80% by weight anatase crystal structure with a minor amount of rutile crystal structure (15% by weight) and a small amount of amorphous phase with approximately 20 nm particle size. Nanoparticles of P25 embedded in nanofiber show high catalytic efficiencies attributed to their phase compositions.

However, several drawbacks of utilizing nanoparticles of titanium dioxide are known. For example, titanium dioxide shows high probability of recombination leading to low rates of the desired chemical transformations with respect to the absorbed light energy. Further, TiO₂ has a relatively large band gap energy and thus requires ultraviolet light for photoactivation. Additionally, TiO₂ exhibits low absorption ability for non-polar organic pollutants. To overcome these drawbacks several strategies and modifications of TiO₂ have been employed. These include extending the wavelength of photoactivation of TiO₂ into the visible region of the spectrum, preventing the electron/hole recombination and/or increasing the adsorption affinity of TiO₂ towards organic pollutants. Modifications of TiO₂ to enhance its photocatalytic properties include metal and non-metal doping, dye sensitization, surface modification as well as fabrication of composites with other materials and immobilization and stabilization on support structures. Examples are TiO₂ nanoparticles loaded with carbon quantum dots (CDs) used to form a composite showing high photoactivity and other good properties for environmental cleaning. TiO₂-Ag composite are known showing slower recombination rates of electron pairs as TiO₂. TiO₂ on zeolite are known as photocatalysts. Further modifications of TiO₂ or other photocatalytic nanoparticles are known.

Photocatalytic composite can comprise a plurality of carriers such as nanofibrous material in form of a mesh, in particular as a non-woven mesh, carrying or doped with photocatalytic nanoparticles. Nanofibers and methods for their production are well known. For example, a nanofibrous medium or nanofibrous membrane can be fabricated by solution-based processing from needle-based electrospinning, nanospider electrospinning and forced/centrifuge spinning. Nanofibers or nanofibrous membrane are decorated with nanoparticles for example via photodeposition process. Another method uses nanoparticles dispersed in a liquid polymeric solution, which will then be formed into electrospun nanofibers. Different types of nanoparticles can be utilized such that they differ in terms of photocatalytic composition, multifunction properties, strength and flexibility.

The effectiveness of photocatalytic filter media is related to the photocatalytic activity of the incorporated photocatalysts. For example, efforts are made to improve the photocatalytic efficiency by reducing the energy bandgap between conduction and valence bands, by increasing the contact area among photocatalyst, target pollutant and light. Furthermore, several strategies for improving the photocatalytic activity are developed for example increasing the light adsorption capacity of the embedded photocatalysts, enhancing visible light activity, retarding the electron-hole pair recombination and increasing the surface area of the photocatalyst. These improvements are related directly or indirectly to the use of nanofibrous material decorated or doped with photocatalytic nanoparticles.

Nanofibers such as hollow nanofibers and solid nanofibers according to its structural characteristics have generally a diameter in the range between 10 and 500 nm. Structures of nanofibers with dimensions inferior to the wavelength of both visible and UV light of 390 nm can generate new functional materials. They are transparent to electromagnetic radiation and therefore allows the entire volume of the tridimensional nanofibrous structure to be correctly irradiated enhancing photocatalytic activity. While a thin layer of nanofibers is able to exhibit good optical transmittance, a thicker layer favors light scattering and enhance luminous efficiency. In disordered mats, non-wovens etc., the inhomogeneous environment at microscales due to the random orientation of nanofibers enhances light-scattering effects.

In photocatalytic systems, the electron-hole pair recombination represents an efficiency limit because the electrons in the conduction band rapidly recombine. For the efficiency of a photocatalyst related to interface-related process it is important to bring the accepting molecules of electrons and those to be decomposed in the vicinity to each other. The ability to capture particles is believed to be due to combination of interception of particles in a nanoscale by the nanofibers as well as the Bownian motion or “random walk” of the particles. Increasing the capture capability may be obtained by increasing the provided surface area such as by reducing the nanofiber diameter and/or by increasing the packing density and/or by providing a turbulence flow for efficient medium diffusion in the reaction system. In the case of a three-dimensional structure made of several layers of nanofibers or nanowires the doped photocatalytic nanoparticles are only a few microns apart from the captured particles. Therefore, the interaction with the electrons generated by the photocatalytic nanoparticles increases significantly by many orders of magnitude.

Generally, a value indicating the photocatalytic activity is the specific surface area expressed in surface-area-to weight ratio in m²/g or the surface-area-to-volume ratio in m²/m³. Photocatalytic nanoparticles or nanocomposites have a very high specific surface area. For example, nanoparticles of TiO₂ with anatase crystal structure have a specific surface area up to 145 m²/g. To prepare a photocatalytic medium such as a photocatalytic membrane, a significant step is to attach the photocatalytic nanoparticles or nanocomposites on an appropriate support such as a membrane or a mesh. Various methods have been investigated such as dip coating, spin coating, electrospinning, physical deposition and immersion precipitation. In particular, electrospinning technique offers an ideal route to construct a nanofiber filtration medium, which is a highly porous nanofiber network structure with controllable pore size and pore size distribution as well as interconnected flow-through pores. Due to the high specific surface area of TiO₂, a photocatalytic nanofiber structure resulting from 1 ml or 1 g of polymeric solution with 10% of nanoparticles of TiO₂ can provide 14.5 m² of photocatalytic surface in a volume of only a few cm³.

Another aspect of using a 3D structure of doped nanofibers is the fact that only a light energy source of low power, which can be integrated into the structure, is necessary to activate the photocatalytic activity of this semiconductor photocatalytic medium even in the bulk of a photocatalytic nanofiber structure. Theoretically, the light energy to excite electrons from semiconductor photocatalytic material has no minimum since a single photon, such as a UV photon, can excite one electron. Therefore, it is possible to utilize only 60 mW of light energy for 1 cm² of a non-woven nanofiber structure with many m² of photocatalytic ability.

### SUMMARY OF THE INVENTION

The object of the present invention is to provide an improved filter medium for air and/or water purification and disinfection. Another aspect of the present invention is to provide a filter unit for the treatment of pollution, which can be used in personal devices and in domestic, transportation and/or industrial environment.

These objects are achieved according to the invention by improving the interaction at an interface between photocatalyst, the target molecule to be destroyed and light. Therefore, the filter medium provides a system that integrates photocatalytic technologies and lightning technologies. Furthermore, according to an improved arrangement between a light energy source and the semiconductor photocatalytic material a unit with high energy efficiency and great compactness, low weight, good portability and wide applicability can be provided.

The solution is based on a new arrangement of a semiconductor photocatalytic material such as nanofibers doped with photocatalytic nanoparticles or at least partially coated with photocatalytic film and a light energy source, radiating light waves in the UV or visible region depending on the type of photocatalytic material applied. According to the invention, the light energy source for radiating UV or visible light is at least partially configured as a support for the semiconductor photocatalytic material. According to the invention, a support can be a structure of a plurality of micro- or nanofibers including optical waveguides or can be a structure of a plurality of photocatalytic micro- or nanofibers showing photocatalytic activity as well as light emitting.

The photocatalyst reaction, which utilizes light as an activating energy source can occur in both of liquid and gas phases. The technology using the photocatalysis reaction with light for redox reaction of organic and/or inorganic molecules is suitable for unspecified decomposition. One of the factors determining the photocatalytic activity of the semiconductor photocatalyst is its light absorption properties. The photocatalytic process takes place when light (photons) having energy equal or greater than the bandgap of the semiconductor photocatalyst is absorbed at the photocatalytic nanoparticle.

The photon transfer can be defined as the efficient irradiation of the photocatalyst with light. Depending on how the lightning source is configured for irradiating the photocatalyst different types of devices or units can be distinguished such with lamps inside or outside the device or with waveguides leading the light from lamps outside the device and/or emitting the light.

Therefore, if the unit is provided to use natural light additional lamps can be used to enhance the performance of the device. Important considerations to improve photocatalytic devices such as the filter medium include mass transfer of target molecule to catalyst surface, maximizing illuminated catalyst surface area, increasing the irradiation amount of surface area per reaction volume that determines the photon transfer rate and enhancing kinetic rate of reaction. Most known photocatalytic materials require high activation energy, which can be found mainly in UV light and is mostly not found in visible light. Therefore, the invention relates to design an improved configuration of a photocatalytic filter medium adapted to use UV light and/or visible light wherein allowing sufficient photon energy to reach the full surface of the catalyst.

According to one embodiment of the invention, the filter medium comprises a structure of at least a plurality of photocatalytic nanofibers doped with or including photocatalytic nanoparticles or coated at least partially with a photocatalytic film. This structure can be in the form of a 3D lattice structure, for example a non-woven mesh or membrane.

This structure can have an influence on the performance of the filter medium due to selective transport of the molecules to be destroyed, products and/or reagents through the structure, in particular a membrane. Beside, providing an efficient way to achieve catalyst recovery, regeneration and reuse by immobilization of photocatalytic material in or on a membrane, the membrane can also define the reaction volume by confining the catalyst, the pollutants and the degradation intermediates into the reaction volume. In a system with immobilized photocatalysts in or on the membrane, the photocatalytic membrane acts both as a selective barrier for the contaminants to be degraded to maintain them in the reaction volume and as a support for the photocatalyst. However, in the case of an immobilized photocatalytic membrane the photocatalyst/reagents contact can be hindered by the mass transfer limitation due to membrane properties.

In a photocatalytic process, the photocatalyst itself is the key component. The properties of the photocatalyst and its concentration in the reaction volume have an important role on photocatalytic performance. Using heterogeneous photocatalytic nanoparticles doped provides multistage photocatalytic reactions and thus results in increasing photocatalytic performance.

Furthermore, the properties of the membrane transport like permeability and selectively are affected by the structure, thickness and composition of the membrane and the layer of photocatalytic nanoparticles. The mass transport through the membrane, in particular the flow rate across the membrane is a key parameter. The flow rate determines the contact times between the photocatalyst and the contaminants as reagents as well as the mass transfer of the contaminants to the catalytic sites and of the degradation products away from them. Therefore, the mass transfer should be fast enough in order to avoid reaction limitation meanwhile the contact time should be appropriate to control reaction selectivity. In the design of photocatalytic structures, not only the selection of an appropriate membrane material is important to provide mechanical, thermal and chemical stability, photostability and transparency, membrane transport properties and the incorporation of the photocatalyst but as well the thickness of the photocatalytic structure.

Increasing the thickness of the photocatalytic structure could bring better results in terms of adsorption capacity and photocatalytic performance due to more electron-hole pairs to be generated. However, beyond a certain value the thickness of the photocatalytic structure has negative impacts on the flow rate across the membrane, the rate of carrier recombination processes and the light intensity entering into the photocatalytic structure. For example, the structure can comprise a plurality of nanofibers made of an optical material, doped with or including photocatalytic nanoparticles or coated at least partially with a photocatalytic film. The optical material can be poly(methyl methacrylate) (PMMA). To provide transportation of photons the nanofibers have a diameter above the wavelength of the stimulating radiation. In one embodiment, the semiconductor photocatalytic material and the light energy source can be provided as an integrated structure wherein a plurality of nanofibers of PMMA constitutes a photocatalytic composite with photocatalytic activity. Furthermore, the integrated structure can comprise a plurality of photocatalytic nanofibers and a plurality of optical micro- or nanofibers embedded.

Alternatively, a plurality of polymeric nanofibers doped with or including photocatalytic nanoparticles or coated at least partially with a photocatalytic film and provided in form of a non-woven mesh or a thin film structure can be applied on various supporting substrates, for example glass, plastics and/or polymer membranes or structures providing not only the support for the semiconductor material but functioning as the light energy source.

In one embodiment, the substrate can be made of an optical material such as poly(methyl-methacrylate) (PMMA), also known as acrylic, acrylic glass or plexiglass. The optical material can be designed in the form of a lattice structure providing a support with defined characteristics and/or an optical fiber delivery system for transmitting and/or emitting light. Alternatively, in the case of photocatalytically coated nanofibers, the optical material can be designed as glass fibers.

According to one embodiment, the light energy source can comprise side emitting or glow fibers or fibrous optical waveguides such that light gradually escapes along the length of fibers creating an even glow. The light energy source receives photons from a visible light or UV light source, which can be arranged inside or outside of a filter unit including the photocatalytic filter medium. This structure combining photocatalytic and lightning activities shows high photodegradation efficiency even with a structure of a certain thickness by overcoming limited light penetration.

The source of light is an important factor in photocatalytic systems as it determines the design and application of the technology. According to one embodiment of the invention, light is emitted from at least one light source such as UV lamp, laser or light-emitting diode (LED). These devices typically emit either longwave (315 to 400 nm) or shortwave (200 to 315 nm) UV radiation. In another embodiment of the invention, visible light can be used as the light energy source, especially when using WO₃ nanoparticles as photocatalyst. One advantage of LEDs lies in the ability, to control the emitted radiant power with the forward current. Normally, low-pressure and medium-pressure mercury lamps are used. Although low-pressure lamps have lower UV intensity in comparison to medium-pressure lamps, their significantly longer service lifetime and lower electrical power input are advantageous.

An efficient light energy source for photocatalysis is one that has uniform light intensity and narrow emission spectra, minimizes heat loss during operation, emits wavelength, which is not absorbed by the substrate/pollutant, provides maximum illumination of the photocatalytic material and is easily incorporated into a filter unit to form a single unit.

In an embodiment of the invention, an electroluminescent (EL) material is used to convert electrical energy into optical energy such as to generate photons. In this case, the light energy source can be configured as a structure of electroluminescent material and is adapted to support the semiconductor photocatalytic material. Electroluminescent materials are able to emit light in response to the application of an electrical current or a strong electric field. Supplying electrical energy to the electroluminescent layer raises luminescent center to its excited state and relaxing to its ground state by dissipating the absorbed energy, for example by emitting a photon. It is known that electroluminescent devices, especially thin film EL devices using organic substances as luminous materials, emit light of high luminance even at low applied voltages and thus show excellent luminous efficiencies. In general, such filter medium comprises a first electrode and a second electrode in form of a mesh and in between layers of the electroluminescent material and the semiconductor photocatalytic material. For example, one of the first electrode or the second electrode can be formed on the electroluminescent layer.

According to an embodiment of the invention, the first electrode and the second electrode, respectively the capacitor plates are provided as an open-mesh grid showing surprisingly similar magnitude to the capacitance of parallel solid-plate condenser even when the surface area of the mesh is less by orders of magnitude then the plate area. The capacitor plates can be formed of electrical insulating strands of filaments which are bonded together to form an open mesh. Metallic coatings disposed on predetermined portions of the mesh, while maintaining the open mesh characteristic can provide the capacitive characteristics. In this case, the photocatalytic nanofibrous structure could be adherent to one of the first and second electrodes or not. Advantageously, by electrostatically charging the mesh small particles like dust particles can be electrostatically captured by the mesh.

Furthermore, a diameter of the nanofibers used as photocatalytic nanofibers can have a diameter in the range between 70 and 700 nm. Photocatalytic material can include titanium dioxide or other well-known component showing photocatalytic properties in form of nanoparticles or as a coating or dispersed in the polymer or as photocatalytic compounds such as TiO₂-Ag, TiO₂-W, CdS-TiO₂. Modified catalysts can be used with antimicrobial activities and photocatalytic activity.

According to an embodiment of the invention, the filter medium can be incorporated into a filter unit equipped with a casing and a light source arranged inside or outside this casing, wherein the light source is adapted to pump in photons to the light energy source. The filter unit including at least a structure of photocatalytic nanofibers doped with or including photocatalytic nanoparticles or at least partially coated with a photocatalytic film and a light energy source. Provided as a single unit this filter unit is characterized by a low pressure drop. The filter medium with a highly porous structure creates a large surface area with an extremely low flow resistance. The filter unit can be arranged in air purifiers, can be used in air circulation systems, personal breath devices and/or stand-alone filtering devices for trapping pollutants and for transforming harmful chemicals and effectively destroy them.

### BRIEF DESCRIPTION OF THE DRAWINGS

Embodiments of the filter medium according to the invention will be explained more closely in the following, by way of example, with reference to the attached drawings:

FIG. 1 schematically illustrates a filter medium according to a first embodiment of the invention; and

FIG. 2 schematically illustrates a filter medium according to a second embodiment of the invention.

### DETAILED DESCRIPTION OF THE INVENTION

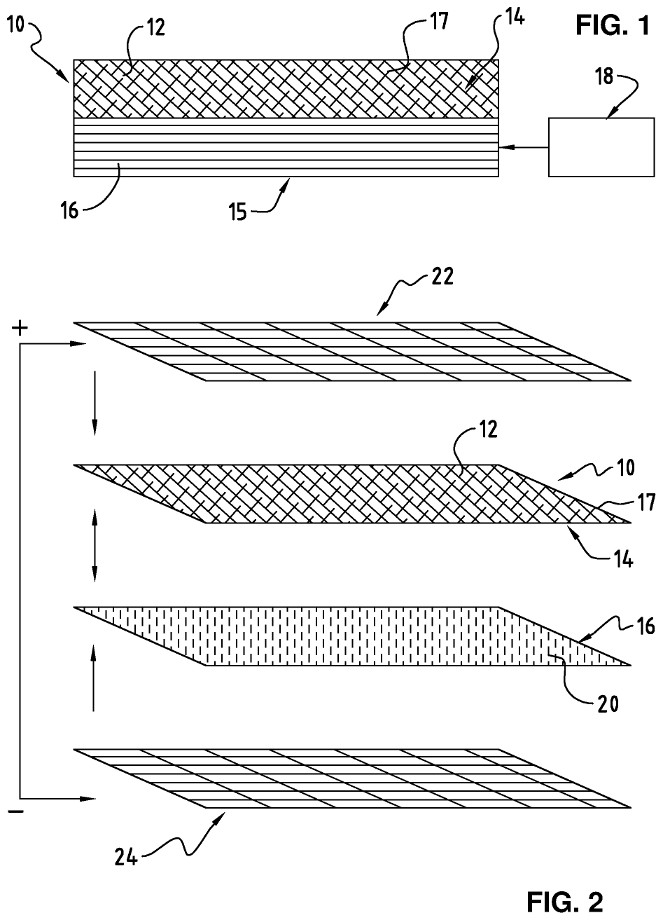

FIG. 1 illustrates a filter medium 10 according to a first embodiment of the invention. The photocatalytic filter medium 10 includes a plurality of photocatalytic nanofibers 12 (shown schematically) doped with or including photocatalytic nanoparticles or coated at least partially with a photocatalytic film. The filter medium 10 can be formed as a non-woven mesh providing a 3D lattice structure. The nanofibers 12 can be provided as polymeric nanofibers on which photocatalytic nanoparticles, for example TiO₂, are decorated. A plurality of the photocatalytic nanoparticles creates photocatalytic surfaces to which water molecules absorbed from a gas or fluid phase can adhere. Due to the hydrophilicity of the TiO₂ water forms an adsorption layer close to the photocatalytic surfaces, which defines a fundamental process occurring at the heterogeneous photocatalytic reaction. Nano-sized TiO₂ has shown enhanced photocatalytic performance due to a large surface to volume ratio or surface to weight ratio. TiO₂ is preferably used in the form of immobilized coating, for example prepared from depositing TiO₂ nanoparticles onto nanofibers 12 or from depositing TiO₂ nanofibers onto a substrate or support.

One possible technique of fabricating network-like three-dimensional nanofibers in a cost-efficient manner is electrospinning. The fabricated nanofibers 12 have a mean diameter of up to 100 nm and the network-like 3D structure functions as carriers for photocatalytic nanoparticles or photocatalytic film.

Furthermore, the purification performance of the filter medium 10 depends on the humidity, the light energy source, the inlet concentration of the pollutants, the type of the photocatalyst and the design of the filter medium.

As shown in FIG. 1, a plurality of nanofibers 12 doped with a photocatalytic compound used as semiconductor material 14 can be configured in form of a structure 17 such as a non-woven mesh or a 3D structure of a predetermined thickness. This structure 17 of semiconductor photocatalytic material 14 can be deposited on a substrate or support 16 made of an optical material. The support can be seen as the light energy source 15 of the filter medium 10. The support 16 can be made of a plurality of fibers made of optical material or optical waveguides such as PMMA fibers. These PMMA fibers can be of side glow type emitting UV photons pumped in by a light source 18 such as a UV light source along the length of the optical waveguides.

FIG. 2 shows another embodiment of the filter medium 10 in a schematic way. As in the first embodiment, the filter medium 10 comprises the structure 17 of a plurality of nanofibers 12 doped with or including photocatalytic nanoparticles. The structure 17 of the semiconductor photocatalytic material 14 is in the form of a non-woven mesh and is deposited on the support 16. According to the second embodiment, the support 16 is made of electroluminescent material 20, in particular in the form of a lattice structure. Both the semiconductor photocatalytic material 14 and the electroluminescent material 20 form layers, which are arranged between a first electrode 22 and a second electrode 24, forming capacitor plates of a condenser device.

In this embodiment, the light energy source is configured as a structure of electroluminescent material 20 and is adapted to support the semiconductor photocatalytic material 14. The electroluminescent material 20 emits light in response to the application of electrical current or a strong electric field, in particular light of high luminance. The embodiment of the filter medium 10 shown in FIG. 2 comprises the first electrode 22 and the second electrode 24 in form of a mesh and in between layers of the electroluminescent material 20 and the semiconductor photocatalytic material 14.

1. Filter medium for air and/or water purification and disinfection, comprising:
a semiconductor photocatalytic material,
a light energy source for radiating light adapted to activate photocatalytic reactions of the semiconductor photocatalytic material, wherein:
the light energy source is configured as a support for the semiconductor photocatalytic material.

2. Filter medium according to claim 1, wherein photocatalytic nanofibers are provided by photocatalytic nanoparticles in or on nanofibers or by a photocatalytic film coated at least partially on nanofibers, wherein a plurality of the photocatalytic nanofibers forms a structure.

3. Filter medium according to claim 2, wherein the photocatalytic nanofibers are made of an optical material.

4. Filter medium according to claim 2, wherein the semiconductor photocatalytic material comprises a non-woven mesh of the photocatalytic nanofibers supported on a substrate of the light energy source made of an optical material.

5. Filter medium according to claim 3, wherein the optical material is poly(methylmethacrylate).

6. Filter medium according to claim 1, wherein the light energy source comprises side glow fibers or fibrous optical waveguides, receiving photons from at least one light source.

7. Filter medium according to claim 6, wherein the at least one light source is a UV light source comprising a UV lamp, a laser or a light emitting diode (LED).

8. Filter medium according to claim 1, wherein the light energy source is configured as a structure of electroluminescent material.

9. Filter medium according to claim 8, wherein the electroluminescent material and the semiconductor photocatalytic material are arranged as layers between a first electrode and a second electrode, which are provided in a form of a mesh, respectively.

10. Filter medium according to claim 2, wherein a diameter of the nanofibers in the range between 70 nm and 700 nm.

11. Filter medium according to claim 1 wherein the semiconductor photocatalytic material includes TiO₂.

12. Filter medium according to claim 1 wherein the semiconductor photocatalytic material includes nanoparticle compounds comprising TiO₂, or TiO₂ decorated Ag or decorated W or decorated WO₃.

13. Filter unit comprising the filter medium according to claim 1, a housing, and at least one light source arranged inside or outside the housing and adapted to pump photons in the light energy source.

14. Filter unit according to claim 13, combined as part of an air circulation system, a personal breath device and/or a stand-alone filtering device.
